(19) United States
(12) Patent Application Publication
(10) Pub. No.: US 2026/0026684 A1
(43) Pub. Date: Jan. 29, 2026

(54) FLEXIBLE PRE-OPERATION ACQUISITION OF OPHTHALMIC IMAGES

(71) Applicant: ALCON INC., Fribourg (CH)

(72) Inventors: Vignesh SURESH, Arlington, TX (US); Lu YIN, Keller, TX (US); Ramesh SARANGAPANI, Coppell, TX (US)

(21) Appl. No.: 19/268,067

(22) Filed: Jul. 14, 2025

Related U.S. Application Data
(60) Provisional application No. 63/676,458, filed on Jul. 29, 2024.

(51) Int. Cl. A61B 3/00; A61B 3/14; G16H 30/20

(52) U.S. Cl. CPC A61B 3/0033; A61B 3/005; A61B 3/14; G16H 30/20

(57) ABSTRACT

A computer-implemented method of acquiring ophthalmic images for clinical use may include receiving, at a user device including a camera, a command to initiate image acquisition relative to an eye of a patient. The computer-implemented method may also include, responsive to the receiving, interactively guiding, at the user device, a user to capture at least one image of the eye that satisfies one or more clinical image parameters. The computer-implemented method may also include, responsive to the interactively guiding, the user device causing the at least one image to be stored in relation to the patient in a location accessible to an ophthalmic surgical system.

### INTRODUCTION

Light received by the human eye passes through the transparent cornea covering the iris and pupil of the eye. The light is transmitted through the pupil and is focused by a crystalline lens positioned behind the pupil in a structure known as a capsular bag. The light is focused by the lens onto the retina, which includes rods and cones capable of generating nerve impulses in response to the light. That crystalline lens may become cloudy, creating a cataract.

Cataract surgeries can treat the above condition by removing the cloudy crystalline lens and replacing it with an intraocular lens (IOL). Cataract surgeries may utilize a toric IOL implantation, where spring-like arms (haptics) are included to hold the IOL in place within the capsular bag. As part of a toric IOL implantation, a surgeon aligns a toric axis of the IOL with the astigmatism axis of the patient's eye. Therefore, in preparation for the toric IOL implantation, the surgeon may visually indicate the astigmatism axis for reference during the implantation. Existing methods of visually indicating the astigmatism axis are typically invasive or require specialized hardware such as an optical biometer.

### SUMMARY

In certain embodiments, one general aspect includes a computer-implemented method of acquiring ophthalmic images for clinical use. The computer-implemented method includes receiving, at a user device including a camera, a command to initiate image acquisition relative to an eye of a patient. The computer-implemented method also includes, responsive to the receiving, at the user device, interactively guiding a user to capture at least one image of the eye that satisfies one or more clinical image parameters. The computer-implemented method also includes, responsive to the interactively guiding, the user device causing the at least one image to be stored in relation to the patient in a location accessible to an ophthalmic surgical system.

In certain embodiments, another general aspect includes a system for capturing acquiring ophthalmic images for clinical use. The system includes a memory having executable instructions, a camera, and a processor in communication with the memory and the camera. The processor is configured to execute the instructions to receive a command to initiate image acquisition relative to an eye of a patient and, responsive to the receipt of the command, to interactively guide a user to capture at least one image of the eye that satisfies one or more clinical image parameters. The processor is further configured to execute the instructions, in response to the interactive guidance, to cause the at least one image to be stored in relation to the patient in a location accessible to an ophthalmic surgical system.

In certain embodiments, another general aspect includes a computer-program product including a non-transitory computer-usable medium having computer-readable program code embodied therein. The computer-readable program code is adapted to be executed to implement a method. The method includes receiving a command to initiate image acquisition relative to an eye of a patient. The method also includes, responsive to the receiving, interactively guiding a user to capture at least one image of the eye that satisfies one or more clinical image parameters. The method also includes, responsive to the interactively guiding, causing the at least one image to be stored in relation to the patient in a location accessible to an ophthalmic surgical system.

### BRIEF DESCRIPTION OF THE DRAWINGS

FIGS. 1A-B illustrate an example of an environment for acquiring ophthalmic images for clinical use, in accordance with certain embodiments of the present disclosure.

FIG. 2A illustrates an example of an ophthalmic image, in accordance with certain embodiments of the present disclosure.

FIG. 2B illustrates an example of a graphically annotated ophthalmic image, in accordance with certain embodiments of the present disclosure.

FIGS. 3A-B illustrate an example of an operating environment for performing ophthalmic procedures, in accordance with certain embodiments of the present disclosure.

FIG. 4 illustrates an example of a process for acquiring and using ophthalmic images, in accordance with certain embodiments of the present disclosure.

FIG. 5 illustrates an example of a process for interactively guiding a user to capture ophthalmic images, in accordance with certain embodiments of the present disclosure.

### DETAILED DESCRIPTION

Currently, various approaches can be used to graphically indicate an ophthalmic attribute of a patient's eye, such as an astigmatism axis, for reference by a surgeon during surgery (e.g., during a toric IOL implantation). Using the astigmatism axis as an example, in some approaches, the astigmatism axis is captured together with a pre-operative image via an optical biometer, and the pre-operative image is then registered with a real-time image of the eye during surgery. According to these approaches, the astigmatism axis can be viewed by the surgeon, during surgery, in relation to the real-time image of the eye. However, such approaches have the disadvantage of requiring the availability of an optical biometer in conjunction with capturing the pre-operative image.

In other approaches, an ophthalmic attribute of the eye can be graphically indicated by a surgeon in the surgical environment. Again using the astigmatism axis as an example, a Mendez degree ring can be placed on the patient's eye and the astigmatism axis can be physically marked, with ink, for reference by the surgeon during surgery. However, this approach is invasive. Furthermore, ink marks are subject to fading, thus necessitating that the astigmatism axis be re-marked in some cases.

The present disclosure describes examples of pre-operatively capturing an ophthalmic image of a patient's eye without utilizing specialized or dedicated equipment such as an optical biometer. In various embodiments, the ophthalmic image can be captured with a user device equipped with a camera, such as a smartphone or tablet, and can be determined to be suitable for use in ophthalmic surgery, for example, in response to a determination that the ophthalmic image is of sufficient quality for registration and clinical use, for example, in ophthalmic surgery. An ophthalmic image that has been deemed to be of sufficient quality for clinical use, for example, in ophthalmic surgery, may be referred to herein as a clinically suitable ophthalmic image, or a “CS-OI.” An ophthalmic image may be deemed to be of sufficient quality for clinical use, for example, as a result of satisfying a configurable set of one or more clinical image parameters, as further discussed below.

In various embodiments, the user device can interactively guide the user to capture the CS-OI based on, for example, the set of one or more clinical image parameter(s). The clinical image parameter(s) can establish a collective quality standard, for example, for registration and use in ophthalmic surgery. The CS-OI can be graphically annotated with an ophthalmic attribute, such as an astigmatism axis, and can be registered with a real-time image of the patient's eye. Accordingly, in certain embodiments, the graphical annotation with the ophthalmic attribute can be overlaid on a real-time display of the patient's eye. Examples will be described relative to the Drawings.

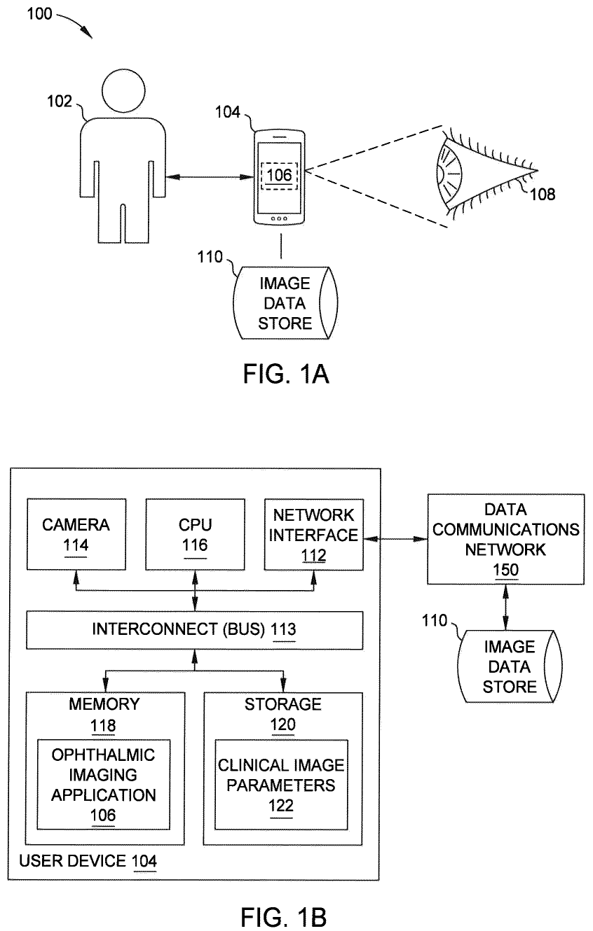

FIGS. 1A-B illustrate an example of an environment 100 for acquiring clinically suitable ophthalmic images (CS-OIs) for clinical use, for example, in ophthalmic surgery (e.g., a toric IOL implantation), in accordance with certain embodiments of the present disclosure. For clarity, FIGS. 1A-B will be described collectively. The environment 100 includes a user device 104, operated by a user 102, and an ophthalmic image data store 110. The user device 104 can be, for example, a smartphone, laptop, tablet, a wearable such as a smartwatch, pin or fitness tracker, and/or the like that may be operable to capture images. In some cases, the user device 104 can be a dedicated camera. For illustrative purposes, the user device 104 is shown to be a smartphone.

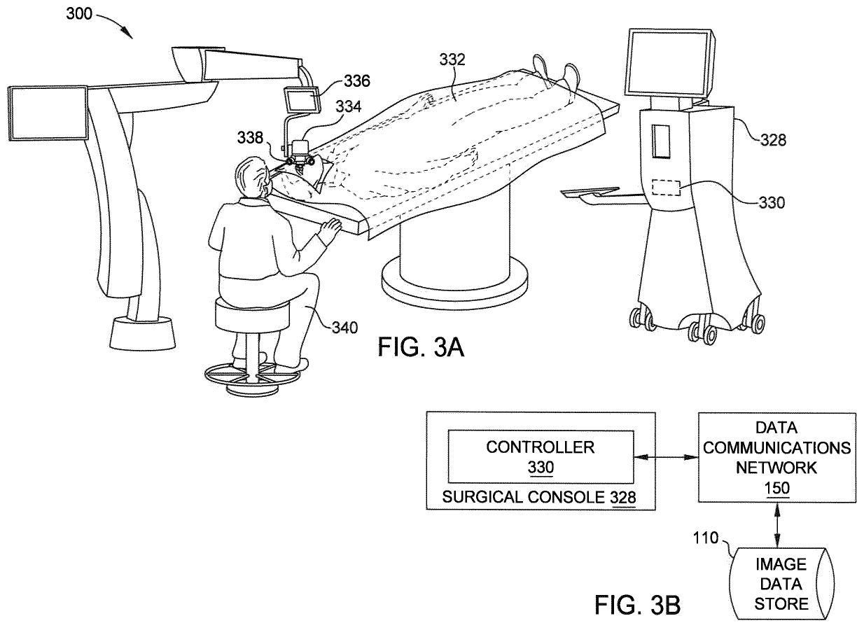

As shown in FIG. 1A, the user device 104 has an ophthalmic imaging application 106 resident and executing thereon. The user device 104, via the ophthalmic imaging application 106, is operable to capture a CS-OI of an eye 108 of a patient, and to store the CS-OI in the ophthalmic image data store 110 in relation to the patient. In various embodiments, the user device 104 can be operated, held, rotated, and/or otherwise manipulated by the user 102 to control an orientation and/or distance thereof relative to the eye 108. In general, the ophthalmic image data store 110 can be a storage location accessible to both the user device 104 and an ophthalmic system that may retrieve the CS-OI for clinical use. The ophthalmic system may be, for example, a surgical console (e.g., surgical console 328) for ophthalmic surgery, and/or a control system (e.g., controller 330) for such a console, as further discussed relative to FIGS. 3A-B.

With reference to FIG. 1B, in some embodiments, the user device 104 and the ophthalmic image data store 110 can communicate over a data communications network 150. The data communications network 150 can be, or can include, one or more of a private network, a public network, a local or wide area network, the Internet, combinations of the same, and/or the like. The data communications network 150 can include, for example, interfaces (e.g., application programming interfaces) for enabling interaction and communication between and among the components and systems of the environment 100 and/or other components and systems. In some embodiments, the ophthalmic image data store 110 can reside on the user device 104. In these embodiments, the user device 104 can access the ophthalmic image data store 110 without relying on the data communications network 150.

The user device 104 can include an interconnect 113 and a network interface 112 for connection with the data communications network 150. The user device 104 can further include a camera 114, a central processing unit (CPU) 116, memory 118, and storage 120. The camera 114 can include any suitable hardware that enables the user device 104 to capture images. In certain embodiments, the camera 114 can correspond to imaging hardware typical of smartphones, smartwatches, tablets, and other mobile devices.

The CPU 116 can retrieve and store application data in the memory 118, as well as retrieve and execute instructions stored in the memory 118. The interconnect 113 transmits programming instructions and application data among the camera 114, the CPU 116, the network interface 112, the memory 118, and the storage 120. The CPU 116 can represent a single CPU, multiple CPUs, a single CPU having multiple processing cores, and the like. The memory 118 represents random access memory. The storage 120 can be a disk drive. Although shown as a single unit, storage 120 can be a combination of fixed or removable storage devices, such as fixed disc drives, removable memory cards or optical storage, network attached storage (NAS), or a storage area-network (SAN).

The storage 120 can include clinical image parameters 122 usable to determine a suitability of an image for clinical use, such as in ophthalmic surgery. The clinical image parameters 122 can include, for example, one or more sets of criteria or logic for determining the suitability. In an example, the clinical image parameters 122 can include a limbus parameter usable by the ophthalmic imaging application 106 to automatically verify a position of a limbus in an image. According to this example, a center of the limbus can be calculated, for example, by performing a segmentation of the image and then calculating a region on either side of the limbus to determine whether the ratio of the two regions is one, or sufficiently close to one according to any suitable measurement. In various embodiments, an offset between a center of the ophthalmic image and the center of the limbus is then measured in any suitable fashion including, for example, pixels, microns, percentage of image height, and/or the like. In various embodiments, the limbus parameter is satisfied if, for example, the offset is less than a threshold offset value.

In another example, the clinical image parameters 122 can include an image blur parameter usable by the ophthalmic imaging application 106 to automatically verify clarity of an image. According to this example, the ophthalmic imaging application 106 can identify an ophthalmic feature in the image, such as one or more blood vessels, and can calculate a variance of a Laplacian transform of an image area including the identified ophthalmic feature(s). In this example, the identified blood vessel(s) can be advantageous ophthalmic features for Laplacian transforms, as they are specific and thin enough to make Laplacian transforms more easily calculable. In various embodiments, the image blur parameter is satisfied if, for example, the variance of the Laplacian transform is less than a predetermined acceptable value that represents image clarity.

In another example, the clinical image parameters 122 can include an image intensity parameter usable by the ophthalmic imaging application 106 to automatically verify that there is sufficient illumination in the image. According to this example, the ophthalmic imaging application 106 can generate an average illumination value for the image or sub-portions within the image and compare the average illumination value to a threshold illumination value. In various embodiments, the image intensity parameter is satisfied if, for example, the average illumination value is at least the threshold illumination value.

In another example, the clinical image parameters 122 can include a visibility parameter usable by the ophthalmic imaging application 106 to automatically verify that one or more selected ophthalmic features, such as pupil and limbus regions, are visible in the image. According to this example, the ophthalmic imaging application 106 can identify the selected ophthalmic features, segment them from the image, and thereby confirm their visibility in the image (e.g., visibility of the pupil and limbus). In various embodiments, the visibility parameter is satisfied if the ophthalmic imaging application 106 is able to identify and segment the selected ophthalmic features from the image. In some embodiments, the ophthalmic imaging application 106 can apply an applicable computer vision standard associated with the ophthalmic images being viewable to a human viewer, such as an ophthalmologist. In these embodiments, the visibility parameter can be satisfied if, for example, the identified and segmented ophthalmic features satisfy the applicable computer vision standard. Other examples will be apparent to one skilled in the art after a detailed review of the present disclosure.

In another example, the clinical image parameters can include a wideness parameter usable to automatically verify that the eye 108, for example, is open sufficiently wide in the image. According to this example, the ophthalmic imaging application 106 can detect eyelid and limbus regions in the image. Thereafter, the ophthalmic imaging application 106 can determine whether the eyelid and limbus regions overlap. In various embodiments, the wideness parameter is satisfied if the eyelid and limbus regions do not overlap in the image, or an amount of overlap is below a threshold amount.

In some embodiments, various combinations of the clinical image parameters 122 may be utilized to determine suitability of an image for clinical use. For example, the clinical image parameters 122 may include the limbus parameter, the blur parameter, the image intensity parameter, the visibility parameter, and the wideness parameter. As another example, the clinical image parameter 122 may include only one of the limbus parameter, the blur parameter, the image intensity parameter, the visibility parameter, or the wideness parameter. As a further example, any combination of the limbus parameter, the blur parameter, the image intensity parameter, the visibility parameter, and the wideness parameter may be included as the clinical image parameters 122.

In some embodiments, the clinical image parameters 122 may be interrelated such that a higher value of one parameter may permit a lower value of another parameter while still being determined suitable (e.g., a higher value of the image intensity parameter may permit a lower value for the visibility parameter). In some embodiments, the clinical image parameters 122 may be weighted such that a more favorable value in one parameter may have a larger impact on suitability than another parameter (e.g., the limbus parameter may be weighted more heavily than the wideness parameter). In some embodiments, one or more, including all, of the clinical image parameters 122 may include a baseline threshold below which the image may not be determined as suitable and a top threshold beyond which the parameter no longer affects suitability (e.g., at values below the baseline threshold for the limbus value, the image is always determined to not be suitable and at values above the top threshold, the limbus value no longer positively affects the determination of suitability, even if the value continues beyond the top threshold).

Memory 118 can be used by the CPU 116 to load and run an operating system and/or one or more applications that operate various aspects of the user device 104. As shown, the memory 118 includes the ophthalmic imaging application 106 discussed above. In various embodiments, the ophthalmic imaging application 106, when executed by the CPU 116, controls the camera 114 to capture ophthalmic images of the eye 108 according to a position of the user device 104. The position can be dynamically controlled by the user 102, for example, as a result of the user 102 holding and aiming the user device 104, or otherwise orienting the camera 114 thereof.

In various embodiments, the ophthalmic imaging application 106 can interactively guide the user 102 to capture a CS-OI. For example, the ophthalmic imaging application 106, in response to a capture command from the user 102, can capture a candidate image of the eye 108. The ophthalmic imaging application 106 can automatically evaluate the suitability of the candidate image for clinical use, for example, in ophthalmic surgery, based on any one or more of the clinical image parameters 122. Thereafter, the ophthalmic imaging application 106 can provide corresponding visual, audible, tactile and/or other feedback to the user 102 related to a determined suitability of the candidate image.

The feedback produced by the ophthalmic imaging application 106 can take various forms. The feedback can include, for example, a prompt indicating that one or more of the clinical image parameters 122 are not satisfied (e.g., a prompt indicating that the limbus, image blur, intensity, visibility and/or wideness parameters are not satisfied), a prompt indicating a suggested adjustment to the user device 104 to achieve suitability (e.g., a suggestion to move or rotate the user device 104 to capture the limbus, to increase illumination of the eye, to pull back the eyelid further, to focus the camera, or any other action), a prompt indicating that all of the clinical image parameters 122, or all of a preconfigured subset thereof, are satisfied (e.g., a prompt indicating that the user device 104, and/or the camera 114 thereof, is suitably positioned to capture the ophthalmic image), combinations of the foregoing and/or the like.

In various embodiments, the ophthalmic imaging application 106 can store the candidate image in the ophthalmic image data store 110, as the CS-OI, in response to a corresponding command from the user 102. The command may follow, for example, favorable feedback relative to the candidate image (e.g., feedback indicating that all of the clinical image parameters 122, or all of a preconfigured subset thereof, are satisfied). In some embodiments, in response to a determination that the candidate image is suitable, the ophthalmic imaging application 106 can automatically store the candidate image in the ophthalmic image data store 110 as the CS-OI.

In some embodiments, as part of interactively guiding the user 102, the ophthalmic imaging application 106 can provide automatic feedback based on a scan of a live field of view of the camera 114. For example, the ophthalmic imaging application 106 can automatically capture a dynamic image of the live field of view and, thereafter, can automatically evaluate the suitability of the dynamic image for clinical use, for example, in ophthalmic surgery. In some embodiments, the dynamic image is used on a temporary basis to automatically assess the position of the user device 104 and is not itself a candidate for clinical use. The ophthalmic imaging application 106 can provide corresponding visual, audible, tactile and/or other feedback to the user 102 related to a determined suitability of the dynamic image, in similar fashion to the feedback described above. In some cases, the ophthalmic imaging application 106 can automatically capture a candidate image of the eye 108 in response to a determination that the dynamic image is suitable. In other cases, the feedback to the user 102 can include a prompt suggesting that the user 102 issue a command to capture a candidate image, as described above. In various embodiments, the ophthalmic imaging application 106 can capture and assess a dynamic image on a regular interval (e.g., every 0.1 seconds, every 0.3 seconds, every 0.5 seconds, every 1 second, every 2 seconds, every 3 seconds, every 4 seconds, every 5 seconds, every 10 seconds, any range bounded by any of the foregoing, etc.).

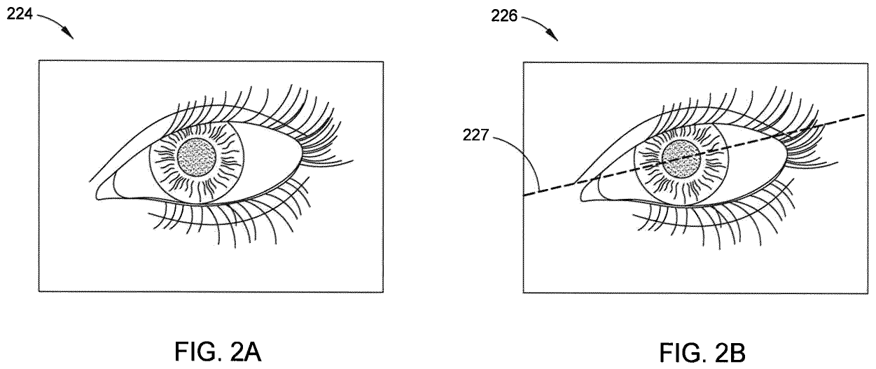

In certain embodiments, the ophthalmic imaging application 106, or another component, can graphically annotate the CS-OI with an ophthalmic attribute of the eye 108, such as an astigmatism axis. Such graphical annotation can occur before and/or after storing the CS-OI. In various embodiments, the graphical annotation can be user-indicated and/or automatically indicated via, for example, artificial intelligence, machine learning, automated image analysis, or any other automatic processing mechanism. The ophthalmic imaging application 106, or another component, can store the graphical annotation in the ophthalmic image data store 110 together with the CS-OI. An example of the graphical annotation will be shown relative to FIG. 2B.

FIG. 2A illustrates an example of an ophthalmic image 224, in accordance with certain embodiments of the present disclosure. In various embodiments, the ophthalmic image 224 may be captured, determined to be suitable, and stored, as discussed relative to FIGS. 1A-B.

FIG. 2B illustrates an example of a graphically annotated ophthalmic image 226, in accordance with certain embodiments of the present disclosure. In particular, the graphically annotated ophthalmic image 226 adds a graphical indication of an astigmatism axis 227 to the ophthalmic image 224 of FIG. 2A. In various embodiments, the astigmatism axis 227 can be added by the ophthalmic imaging application 106, or another component, in response to a user indication and/or in response to an automatic indication via, for example, artificial intelligence, machine learning, automated image analysis, or any other automatic processing mechanism.

FIGS. 3A-B illustrate an example of an operating environment 300 for performance of an ophthalmic procedure, such as a toric IOL implantation, in accordance with certain embodiments of the present disclosure. For clarity, FIGS. 3A-B will be described collectively. As shown, the operating environment 300 includes a surgeon 340, a patient 332, as well as a plurality of surgical systems and devices, such as a surgical console 328, a microscope system 334, and a display 336. The microscope system 334 can include binoculars 338.

The surgical console 328 includes a controller 330. In the example of FIGS. 3A-B, the controller 330 is integrated within the surgical console 328, where the controller 330 includes or refers to one or more processors and/or memory devices integrated within the surgical console 328. In certain other embodiments, the controller 330 is a stand-alone device or module that is in wireless or wired communication with, e.g., the surgical console 328, the microscope system 334, and other devices within the operating environment 300. In certain embodiments, the controller 330 refers to a set of software instructions that a processor associated with the surgical console 328 is configured to execute. In certain aspects, operations of the controller 330 may be executed partly by the processor associated with controller 330 and/or the surgical console 328 and partly in a public or private cloud. The controller 330 is operable to communicate over the data communications network 150 described relative to FIG. 1B.

In the context of the operating environment 300, the patient 332 may be, for example, the patient corresponding to the eye 108 of FIGS. 1A-B. The controller 330 can retrieve the pre-operative CS-OI of the eye 108, and the graphical annotation thereof, from the ophthalmic image data store 110. In some embodiments, the ophthalmic image data store 110 can reside on the surgical console 328 and/or the controller 330. Thereafter, the controller 330 can cause real-time information to be presented to the surgeon 340, for example, via the binoculars 338 of the microscope system 334 and/or via the display 336. The presented information can include a real-time image of the eye 108 overlaid with, for example, the graphical annotation of the ophthalmic attribute (e.g., astigmatism axis) of the eye 108, as indicated by the pre-operative CS-OI and discussed above relative to FIGS. 1A-B and 2A-B. In various embodiments, the information presented to the surgeon 340 can be based on a registration of the CS-OI of the eye 108 with the real-time image of the eye 108. For example, the CS-OI may be registered based on iris features, retinal blood vessels, or other registration techniques. For example, one or more landmarks of the iris or blood vessels may be identified and aligned in both the CS-OI and the real-time image of the eye 108 to align the two images. In various embodiments, the surgeon 340 can thereby be provided actionable information for the ophthalmic surgery. For example, as discussed relative to FIGS. 1A-B and 2A-B, the surgeon 340 can be provided the graphical annotation of the astigmatism axis for accurate alignment thereof with a toric axis of a toric IOL.

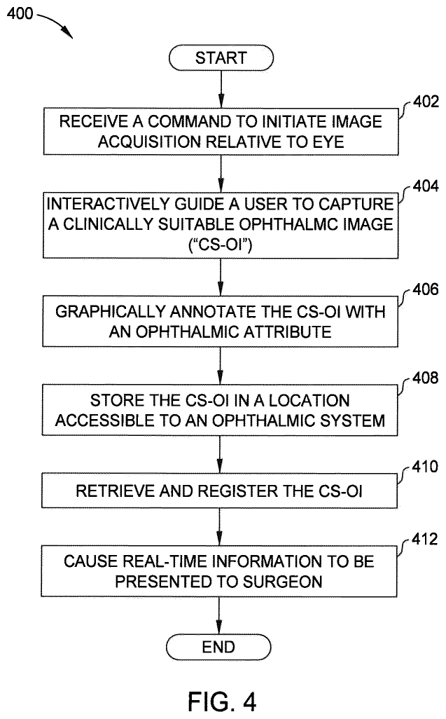

FIG. 4 illustrates an example of a process 400 for acquiring and using CS-OIs, in accordance with certain embodiments of the present disclosure. In certain embodiments, the process 400 can be implemented by any system that can process images. Although any number of systems, in whole or in part, can implement the process 400, to simplify discussion, the process 400 will be described in relation to example components described relative to FIGS. 1A-B and/or 3A-B.

At block 402, the user device 104 receives a command to initiate image acquisition relative to an eye of a patient. The block 402 can include, for example, the user 102 opening and using the ophthalmic imaging application 106 of FIGS. 1A-B.

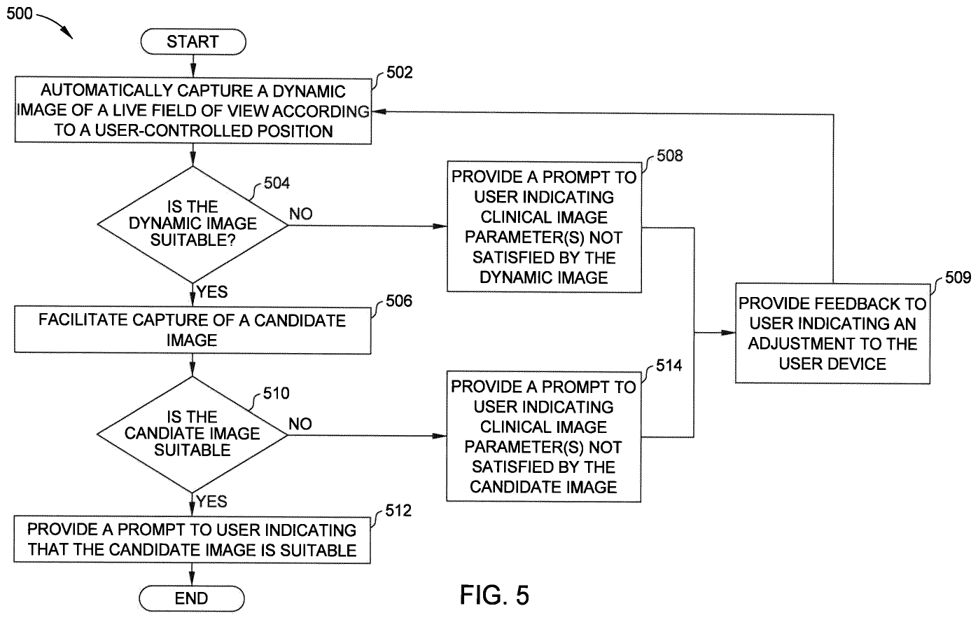

At block 404, the user device 104 interactively guides the user 102 to capture a CS-OI. As discussed relative to FIGS. 1A-B, interactively guiding the user 102 can include, for example, the ophthalmic imaging application 106 capturing a candidate image of the eye 108, automatically determining a suitability of the candidate image, and providing feedback to the user 102 related to the determined suitability. In addition, or alternatively, interactively guiding the user 102 can include, for example, the ophthalmic imaging application 106 automatically capturing and assessing a dynamic image of a live field of view of the camera 114, as discussed relative to FIGS. 1A-B. Examples of functionality that can be performed at the block 404 will be described with respect to FIG. 5.

At block 406, the user device 104, or another component, graphically annotates the CS-OI with an ophthalmic attribute. The ophthalmic attribute can be, for example, an astigmatism axis as discussed relative to FIGS. 1A-B and 2B. In various embodiments, the ophthalmic attribute can be user-indicated. For example, the user 102 can draw the ophthalmic attribute on the CS-OI (e.g., a line). In various embodiments, the ophthalmic attribute can be automatically indicated. For example, rules or artificial intelligence can identify a location of the ophthalmic attribute in the CS-OI and can represent the ophthalmic attribute accordingly (e.g., a line). At block 408, the user device 104 stores, or causes storage of, the CS-OI together with the graphical annotation of the ophthalmic attribute, in the ophthalmic image data store 110 in relation to the patient.

Blocks 410 and 412 relate to use of the CS-OI in ophthalmic surgery. At block 410, the controller 330 retrieves the CS-OI of the eye 108, and the graphical annotation thereof, from the ophthalmic image data store 110, and registers the retrieved CS-OI with a real-time image of the eye 108. At block 412, the controller 330 causes real-time information to be presented to the surgeon 340, for example, via the binoculars 338 of the microscope system 334 and/or via the display 336, as discussed relative to FIGS. 3A-B. The presented information can include the real-time image of the eye 108 overlaid with, for example, the graphical annotation of the ophthalmic attribute of the eye 108 (e.g., the astigmatism axis), as discussed above relative to FIGS. 3A-B. After block 412, the process 400 ends.

FIG. 5 illustrates an example of a process 500 for interactively guiding a user to capture a CS-OI, in accordance with certain embodiments of the present disclosure. The process 500 can be performed, for example, as all or part of the block 404 of FIG. 4. In certain embodiments, the process 500 can be implemented by any system that can process images. Although any number of systems, in whole or in part, can implement the process 500, to simplify discussion, the process 500 will be described in relation to example components described relative to FIGS. 1A-B.

At block 502, the ophthalmic imaging application 106 automatically captures a dynamic image of a live field of view of the camera 114, for example, according to a current user-controlled position of the user device 104 and/or the camera 114 thereof, as discussed relative to FIGS. 1A-B.

At decision block 504, the user device 104 determines whether the dynamic image is suitable for clinical use, for example, based on any one or more of the clinical image parameters 122, as discussed relative to FIGS. 1A-B. If it is determined, at the decision block 504, that the dynamic image is suitable for clinical use, the process 500 proceeds to block 506. If it is determined, at the decision block 504, that the dynamic image is not suitable for clinical use, the process proceeds to block 508. In some embodiments, blocks 502 and 504 can be omitted, such that there is no automatic capture and suitability analysis of dynamic images.

At block 506, the ophthalmic imaging application 106 facilitates capture of a candidate image of the eye 108 for clinical use. For example, as discussed relative to FIGS. 1A-B, in some cases, the block 506 can include the ophthalmic imaging application 106 automatically capturing the candidate image responsive to the determination that the dynamic image is suitable. In other cases, as also discussed relative to FIGS. 1A-B, the ophthalmic imaging application 106 can provide feedback, such as an audible, visible and/or tactile prompt, suggesting that the user 102 issue a command to capture the candidate image. In such cases, the block 506 can include the ophthalmic imaging application 106 receiving the command to capture the candidate image responsive to the prompt. In still other cases, such as embodiments where the blocks 502 and 504 are omitted, the ophthalmic imaging application 106 can enable the user 102 to issue a command to capture the candidate image without any corresponding prompt or prior feedback having been provided.

At block 508, the ophthalmic imaging application 106 provides a prompt to the user 102 indicating that one or more of the clinical image parameters 122 are not satisfied by the dynamic image (e.g., a prompt indicating that the limbus, image blur, intensity, visibility and/or wideness parameters are not satisfied). At block 509, the ophthalmic imaging application 106 may provide feedback to the user 102 indicating an adjustment to the user device 104 based on the one or more of the clinical image parameters 122 that are not satisfied, so as to increase a likelihood that a subsequently captured image will be suitable. The feedback may include, for example, a suggestion to move or rotate the user device 104 to capture the limbus, to increase illumination of the eye, to pull back the eyelid further, to focus the camera, or any other action. From block 509, the process 500 returns to block 502 and executes as described previously.

At decision block 510, the ophthalmic imaging application 106 determines whether the candidate image is suitable for clinical use, for example, based on the clinical image parameters 122, as discussed relative to FIGS. 1A-B. If it is determined, at the decision block 510, that the candidate image is suitable for clinical use, the process may proceed to the block 512. If it is determined, at the decision block 510, that the candidate image is not suitable for clinical use, the process may proceed to the block 514.

At block 512, the ophthalmic imaging application 106 provides a prompt to the user 102 indicating that the clinical image parameters 122 are satisfied. As discussed relative to FIGS. 1A-B, in various embodiments, the ophthalmic imaging application 106 can store the candidate image in the ophthalmic image data store 110, as the CS-OI, in response to a corresponding command from the user 102. In some embodiments, in response to the determination that the candidate image is suitable, the ophthalmic imaging application 106 can automatically store the candidate image in the ophthalmic image data store 110 as the CS-OI. After block 512, the process 500 ends.

At block 514, the ophthalmic imaging application 106 provides a prompt to the user 102 indicating that one or more of the clinical image parameters 122 are not satisfied by the candidate image. From block 514, the process 500 proceeds to block 509. At block 509, as discussed above, the ophthalmic imaging application 106 may provide feedback to the user 102 indicating an adjustment to the user device 104 based on the one or more of the clinical image parameters 122 that are not satisfied, so as to increase a likelihood that a subsequently captured image will be suitable. The feedback may include, for example, a suggestion to move or rotate the user device 104 to capture the limbus, to increase illumination of the eye, to pull back the eyelid further, to focus the camera, or any other action. From block 509, the process 500 returns to block 502 and executes as described previously.

As used herein, a phrase referring to “at least one of” a list of items refers to any combination of those items, including single members. As an example, “at least one of: a, b, or c” or “at least one of: a, b, and c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. A computer-implemented method of acquiring ophthalmic images for clinical use, the method comprising:
receiving, at a user device comprising a camera, a command to initiate image acquisition relative to an eye of a patient;
responsive to the receiving, at the user device, interactively guiding a user to capture at least one image of the eye that satisfies one or more clinical image parameters; and
responsive to the interactively guiding, the user device causing the at least one image to be stored in a location accessible to an ophthalmic surgical system.

2. The computer-implemented method of claim 1, wherein the interactively guiding comprises:
capturing a first image of a field of view of the camera;
determining suitability of the first image for clinical use relative to the eye based on the one or more clinical image parameters; and
providing a prompt to the user related to a result of the determining suitability.

3. The computer-implemented method of claim 2, wherein:
the determining suitability comprises determining that the first image is unsuitable for clinical use relative to the eye based on a determination that at least one clinical image parameter is not satisfied; and
the prompt indicates that the at least one clinical image parameter is not satisfied.

4. The computer-implemented method of claim 3, further comprising providing, to the user, feedback indicating a suggested adjustment to the user device based on the at least one clinical image parameter that is not satisfied.

5. The computer-implemented method of claim 2, wherein:
the determining a suitability comprises determining that the first image is suitable for clinical use relative to the eye based on a determination that the one or more clinical image parameters are satisfied; and
the prompt indicates that the one or more clinical image parameters are satisfied.

6. The computer-implemented method of claim 5, wherein the at least one image comprises the first image.

7. The computer-implemented method of claim 5, wherein:
the first image comprises a dynamic image automatically captured by the user device according to a user-controlled position of the camera; and
the at least one image comprises a second image that is automatically captured by the user device responsive to the determining that the first image is suitable for clinical use.

8. The computer-implemented method of claim 5, wherein:
the first image comprises a dynamic image automatically captured by the user device according to a user-controlled position of the camera;
the interactively guiding further comprises receiving a capture command from the user responsive to the prompt; and
the at least one image comprises a second image captured by the user device responsive to the capture command.

9. The computer-implemented method of claim 2, wherein the determining a suitability comprises automatically verifying that an offset between a center of the first image and a center of a limbus is less than a threshold offset value.

10. The computer-implemented method of claim 2, wherein the determining a suitability comprises automatically verifying a clarity of the first image based on a Laplacian transform of an image area including one more blood vessels.

11. The computer-implemented method of claim 2, wherein the determining a suitability comprises automatically verifying an image intensity of the first image based on a comparison of an average illumination of the image to a threshold illumination value.

12. The computer-implemented method of claim 2, wherein the determining a suitability comprises automatically verifying a visibility of limbus and pupil regions in the first image.

13. The computer-implemented method of claim 2, wherein the determining a suitability comprises automatically verifying that the eye is open sufficiently wide in the first image based on a determination that an eyelid and a limbus do not overlap.

14. The computer-implemented method of claim 2, wherein the first image is captured in response to a capture command from the user.

15. The computer-implemented method of claim 2, wherein the first image comprises a dynamic image automatically captured by the user device according to a user-controlled position of the camera.

16. The computer-implemented method of claim 1, further comprising graphically annotating the at least one image with an ophthalmic attribute, the causing the at least one image to be stored comprising causing information related to the graphically annotating to be stored together with the at least one image.

17. The computer-implemented method of claim 16, wherein the ophthalmic attribute comprises an astigmatism axis of the eye of the patient.

18. A system for capturing acquiring ophthalmic images for clinical use, the system comprising:
a memory comprising executable instructions;
a camera;
a processor in communication with the memory and the camera and configured to execute the instructions to:
receive a command to initiate image acquisition relative to an eye of a patient;
responsive to the receipt of the command, interactively guide a user to capture at least one image of the eye that satisfies one or more clinical image parameters; and
responsive to the interactively guiding, cause the at least one image to be stored in relation to the patient in a location accessible to an ophthalmic surgical system.

19. The system of claim 18, wherein the interactive guidance comprises:
capturing a first image of a field of view of the camera;
determining a suitability of the first image for clinical use relative to the eye based on the one or more clinical image parameters; and
providing a prompt to the user related to a result of the determining suitability.

20. The system of claim 18, wherein the processor is further configured to execute the instructions to graphically annotate the at least one image with an ophthalmic attribute, the storage of the at least one image comprising storage of information related to the graphical annotation together with the at least one image.

21. A computer-program product comprising a non-transitory computer-usable medium having computer-readable program code embodied therein, the computer-readable program code adapted to be executed to implement a method comprising:
receiving a command to initiate image acquisition relative to an eye of a patient;
responsive to the receiving, interactively guiding a user to capture at least one image of the eye that satisfies one or more clinical image parameters; and
responsive to the interactively guiding, causing the at least one image to be stored in relation to the patient in a location accessible to an ophthalmic surgical system.
